(19) United States
(12) Patent Application Publication
(10) Pub. No.: US 2026/0283524 A1
(43) Pub. Date: Sep. 24, 2026

(54) ULTRA-CONFORMAL SKIN-INTERFACED SENSING PLATFORM FOR MOTION ARTIFACT-FREE MONITORING

(71) Applicant: The Penn State Research Foundation, University Park, PA (US)

(72) Inventors: Huanyu Cheng, University Park, PA (US); Yuyan Gao, University Park, PA (US)

(73) Assignee: The Penn State Research Foundation, University Park, PA (US)

(21) Appl. No.: 19/569,435

(22) Filed: Mar. 17, 2026

Related U.S. Application Data
(60) Provisional application No. 63/776,453, filed on Mar. 24, 2025.

(51) Int. Cl. A61B 5/268; A61B 5/28; A61B 5/291; A61B 5/296; B23K 26/364; B23K 101/38; B23K 103/16

(52) U.S. Cl. CPC A61B 5/268; A61B 5/28; A61B 5/291; A61B 5/296; B23K 26/364; A61B 2562/125; B23K 2101/38; B23K 2103/16

(57) ABSTRACT

Embodiments relate to skin-interfaced bioelectronic sensing platforms and methods for motion artifact-free monitoring of physiological signals, including electrophysiological, biochemical, and biophysical parameters, using conformal, water-deformable electrodes. A method of forming the electrodes includes preparing a nanocomposite solution comprising nanoparticles, such as core-shell nanoparticles, dispersed within a polymeric solution; depositing the nanocomposite solution onto a substrate to form a film; patterning the film via laser scribing to form the electrode; and optionally heating the electrode.

### CROSS-REFERENCE TO RELATED APPLICATIONS

This application claims priority to U.S. Provisional Patent Application No. 63/776,453, which was filed on Mar. 24, 2025. The entirety of this application is incorporated by reference herein.

### STATEMENT REGARDING FEDERALLY SPONSORED RESEARCH DEVELOPMENT

This invention was made with government support under Grant Nos. 2319139, ECCS2222654 and ECCS2309323 awarded by the National Science Foundation, under Grant Nos. EB030140 and DA056242 awarded by the National Institutes of Health and under Grant No. OH012220 awarded by the Center for Disease Control and Prevention. The Government has certain rights in the invention.

### FIELD

The present disclosure generally relates to skin-interfaced bioelectronic sensing platforms and methods for motion artifact-free monitoring of physiological signals, including electrophysiological, biochemical, and biophysical parameters, using conformal, water-deformable electrodes.

### BACKGROUND

Skin-interfaced bioelectronics have emerged as a promising class of devices for health monitoring and disease management. These platforms are capable of collecting physiological signals and biochemical information, which can be utilized for early diagnosis of health conditions, as well as for providing stimulation for drug delivery and therapeutic interventions. The integration of electronics with the skin offers a minimally invasive approach to continuous health monitoring, with potential applications spanning wearable health devices, personalized medicine, and remote patient care.

Despite significant advancements, a key challenge in the field remains the interference caused by motion artifacts resulting from body movements and natural skin motions such as stretching, compression, and bending. These artifacts can significantly degrade the accuracy and reliability of the signals obtained, complicating data interpretation and clinical decision-making. Various post-processing strategies have been explored to mitigate noise and artifacts; however, these methods often risk filtering out useful information and are not universally applicable across different device configurations or use cases.

To address these issues, research has focused on developing flexible, stretchable, and conformal bioelectronic devices that can deform with the skin, thereby reducing motion artifacts at the source. Approaches include the use of strain isolation techniques, stretchable conductors, and multiple sensor arrays designed for post-processing correction. Nonetheless, many of these solutions rely on complex, high-cost manufacturing processes, such as multi-step chemical synthesis, or require specialized, device-specific designs and integrated semiconductor components. Consequently, there remains a high demand for simple, universal, low-cost, and scalable materials and sensing platforms capable of providing motion artifact-free monitoring of a broad range of biophysical and biochemical signals.

### SUMMARY

We have developed an ultra-conformal, water-deformable sensing device through a straightforward and scalable laser patterning process. The laser scribing process enables a versatile fabrication method that can be used to produce a range of wearable electrodes.

The core of the device is a highly conductive and stretchable electrode based on room-temperature coalesced nanoparticles, such as core-shell nanoparticles, embedded within a water-responsive polymer matrix. This electrode is capable of partially dissolving and deforming upon contact with moisture, allowing it to form a conformal contact with the skin even during motion or deformation. Moreover, the water-removable feature of the device ensures its safe application on delicate skin, such as that of infants and the elderly, facilitating ease of removal and reducing discomfort.

The resulting sensing devices demonstrate enhanced electrophysiological signal quality and robustness against mechanical disturbances, making them suitable for continuous health monitoring and human-machine interface applications, among other uses. The device can also incorporate commercial off-the-shelf (COTS) chips to extend its functionalities.

In an exemplary embodiment, a method of forming an electrode includes preparing a nanocomposite solution comprising nanoparticles dispersed within a polymeric solution; depositing the nanocomposite solution onto a substrate to form a film; patterning the film via laser scribing to form the electrode; and optionally heating the electrode.

In some embodiments, the nanoparticles are core-shell nanoparticles comprising a core formed from a first metal and a shell formed from a second metal. The first metal and the second metal are selected from the group consisting of silver, gold, platinum, palladium, titanium, copper, and zinc.

In some embodiments, the first metal is silver and the second metal is copper.

In some embodiments, the polymeric solution includes one or more polymeric components selected from the group consisting of polyvinyl alcohol, ethylene vinyl alcohol, polyvinylpyrrolidone, polyethylene glycol, vinyl acetate ethylene, poly(acrylic acid), polyacrylic acid sodium salt, carboxymethyl cellulose, hydroxypropyl methylcellulose, and mixtures thereof.

In some embodiments, the polymeric solution includes polyvinyl alcohol and polyethylene glycol.

In some embodiments, the polymeric solution further includes one or more of the following: one or more plasticizers, one or more lubricants, one or more humectants, and one or more reinforcing fillers.

In some embodiments, the polymeric solution further includes one or more components selected from the group consisting of glycerol, propylene glycol, ethylene glycol, sorbitol, polyethylene glycol, xylitol, and mannitol; and one or more components selected from the group consisting of montmorillonite, kaolin, halloysite nanotubes, laponite, and silica nanoparticles.

In some embodiments, the polymeric solution further includes glycerol and montmorillonite.

In some embodiments, the nanocomposite solution includes 50-90 wt % of the nanoparticles, based on the total weight of the nanocomposite solution.

In an exemplary embodiment, an electrode is formed from the method described above.

In some embodiments, one or more components of a polymeric matrix partially dissolves in the presence of water.

In some embodiments, the electrode has a sheet resistance at or below 1.2 Ω/sq upon stretching the electrode up to 30%.

In an exemplary embodiment, a system includes a sensing device including at least one electrode as described above, wherein the at least one electrode is configured to collect electrophysiological data; and an input/output device configured to receive the electrophysiological data from the sensing device.

In some embodiments, the system further includes a central computer device configured to receive the electrophysiological data from the sensing device and/or from the input/output device.

In some embodiments, the electrophysiological data includes one or more selected from the group consisting of electrocardiograms (ECG) signals, electromyograms (EMG) signals, and electroencephalograms (EEG) signals.

In an exemplary embodiment, a system includes a sensing device including at least one electrode as described above, wherein the at least one electrode is configured to collect data; and an input/output device configured to receive the data from the sensing device. The data is selected from the group consisting of temperature data, humidity data, pulse rate, blood oxygen saturation, and combinations thereof.

In some embodiments, the system further includes a central computer device configured to receive the data from the sensing device and/or from the input/output device.

Further features, aspects, objects, advantages, and possible applications of the present disclosure will become apparent from a study of the exemplary embodiments and examples described below, in combination with the Figures, and the appended claims.

### BRIEF DESCRIPTION OF THE DRAWINGS

The above and other objects, aspects, features, advantages and possible applications of the present innovation will be more apparent from the following more particular description thereof, presented in conjunction with the following drawings. Like reference numbers used in the drawings may identify like components.

FIG. 1 is a flow chart demonstrating an exemplary method of fabricating an electrode.

FIG. 2 is a schematic illustration of an exemplary fabrication process.

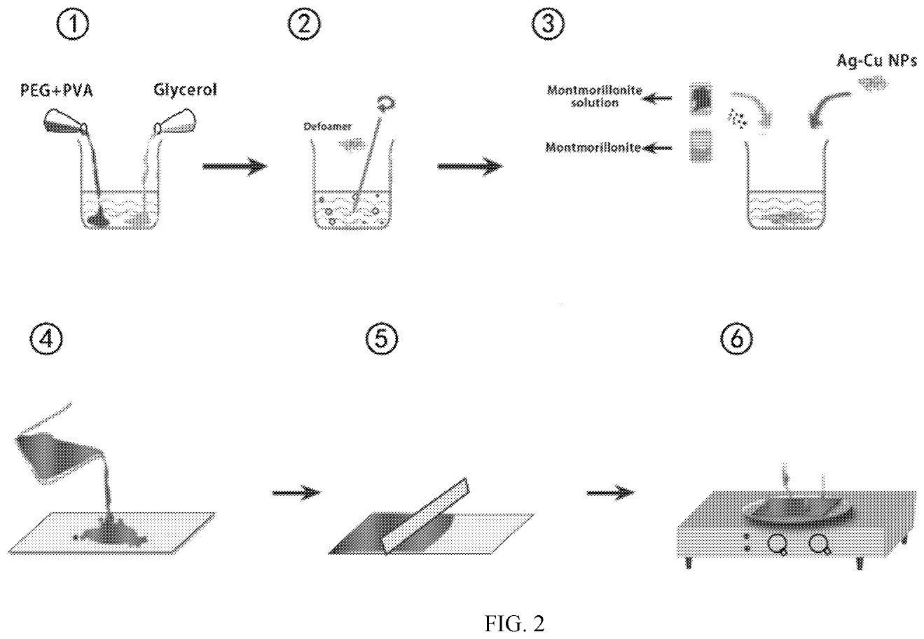

FIG. 3 is a schematic illustration (left) and demonstration (right) of a conformal stretchable device platform.

FIG. 4 is a schematic illustration showing the mechanism of an ultra-conformal device/skin interface from partial dissolution and local deformation assisted by water molecules.

FIG. 5 is a graph showing the low contact impedance of a conformal stretchable thin electrode (80 wt % Ag—Cu NPs).

FIG. 6A is a schematic block diagram illustrating an exemplary embodiment of a system for transmitting data collected by an electrode.

FIG. 6B is a schematic block diagram illustrating an exemplary embodiment of a system or platform for transmitting data collected by more than one electrode.

FIG. 6C is a schematic block diagram of an exemplary embodiment of a system or platform utilizing a plurality of electrodes for transmitting data to at least one input/output device for collection and evaluation of the data. The collected data can be evaluated by the input/output device or a central computer device that can be communicatively connected to the input/output device.

FIG. 7 is a graph showing low sheet resistance of a conformal stretchable thin electrode (80 wt % Ag—Cu NPs).

FIG. 8 is a graph showing the high electromechanical performance of a conformal stretchable thin electrode (80 wt % Ag—Cu NPs).

FIG. 9 includes photographs of a wax-based finger mold and a conformed stretchable thin electrode with the corresponding fingerprint pattern.

FIG. 10 includes an optical profilometer image (left) and comparison analysis (right) of the surface morphology between the two.

FIG. 11 is a schematic illustration showing the modeling of the contact impedance at the electrode-skin interface.

FIG. 12 is a graph showing skin-electrode contact impedance versus frequency for sensors with different sizes.

FIG. 13 is a graph showing a comparison of the contact impedance between the conformal stretchable and commercial gel electrodes with and without external perturbations.

FIG. 14 shows SEM images of electrodes with different Ag—Cu NPs concentrations (60, 65, 70, 75, and 80 wt %).

FIG. 15 is a graph showing the sheet resistance of the thin electrode with increasing Ag—Cu NPs concentration from 60 to 80 wt %.

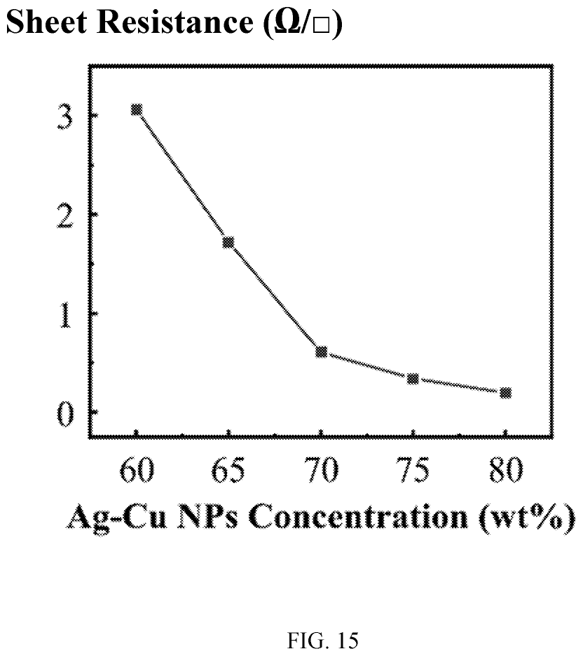

FIG. 16 includes (top-left) a schematic illustration showing the three-lead measurement of the ECG signals upon (top-right) finger pressing, (bottom-left) finger squeezing, and (bottom-right) nearby pen pressing.

FIG. 17 shows a comparison of ECG signals between a conformal stretchable electrode and a commercial gel electrode in the time and frequency domains. ECG signals in the time domain for (top) finger pressing and (bottom) finger squeezing.

FIG. 18 shows a comparison of ECG signals between a conformal stretchable electrode and a commercial gel electrode in time and frequency domains. ECG signals before motions in the frequency domain for (top) finger pressing and (bottom) finger squeezing.

FIG. 19 shows a comparison of ECG signals between a conformal stretchable electrode and a commercial gel electrode in the time and frequency domains. ECG signals after motions in the frequency domain for (top) finger pressing and (bottom) finger squeezing.

FIG. 20 shows a comparison of facial EMG signals between the conformal stretchable and commercial gel electrodes.

FIG. 21 shows an optical image showing the measurement location (left) and the corresponding measured EMG signals from the finger lumbrical muscle before and after exerting the force at the fingertip (right).

FIG. 22 shows a comparison of EMG signals between the conformal stretchable and commercial gel electrodes obtained from the lumbrical muscle of fingers during relaxing and stretching.

FIG. 23 shows a comparison of deformation effects on physiological signal detection performance under poking and bending for commercial electrodes and present electrodes. The error bars presented were based on standard deviation.

FIG. 24 shows an infrared image of the heater on a glass slide (left) and the dependence of the temperature on the applied voltage (middle), along with the on-body demonstration validated against a commercial thermometer (right). The error bars were based on the standard deviation from three measurements.

FIG. 25 is a graph showing the dependence of the output peak temperature of the on-skin spiral-shaped heater on the input power.

FIG. 26 is a graph showing the capacitance change of the humidity sensor as a function of human hydrous exhaling at different durations. The error bars presented in the figures were based on the standard deviation from three measurements.

FIG. 27 is a graph showing the relationship between the capacitance of the humidity sensor and the humidity level. The error bars presented in the figure were based on the standard deviation from three measurements.

FIG. 28 is a graph showing the measured pulse rate and SpO₂ level from the integrated oximeter at the fingertip before and after the 2-minute exercise.

FIG. 29 is a graph showing human-machine interface based on the EMG electrodes to control the game character with bimanual gestures.

FIG. 30 shows eye movements (looking up, down, left, right, and blinking) captured by EOG signals from the conformal stretchable electrode.

FIG. 31 shows graphs demonstrating the changes in electrical resistance from skin deformation during various arm gestures: sensor on the fingers upon making fists (left) and sensor on the wrist upon wrist rotation (right).

FIG. 32 shows (left) an experimental setup and (right) the collected brain wavelength contour from the in-ear EEG sensor consisting of the conformal stretchable patterned thin film on a commercial deformable foam earplug in thinking mode (0-1.5 minutes) and rest mode (1.5-3 minutes).

FIG. 33 shows EEG signals collected from various earplugs across different individuals.

FIG. 34 shows an optical image of tripolar concentric electrodes placed on the ear and corresponding EEG measurements for capturing alpha waves between eye opening and closing.

FIG. 35 shows an image sequence to show the device removal from the human skin.

FIG. 36 shows optical images showing the tensile test of the material: 0 (left), 40 (middle), and 100% stretching (right).

FIG. 37 includes a schematic illustration and optical images of the testing setup to measure (a) EMG, (b) EEG, (c) ECG, and (d) RH %.

### DETAILED DESCRIPTION

The following description illustrates exemplary embodiments and methods of use that are presently contemplated for implementing the present invention. This description is not intended to be limiting, but rather to elucidate the general principles and features of various aspects of the invention. The scope of the invention is not restricted by this description.

Embodiments relate to conformal, water-deformable electrodes that may be incorporated into skin-interfaced bioelectronic sensing devices and platforms. The electrodes may specifically serve as sensors configured to acquire high-quality physiological signals in a manner that is resistant to motion artifacts. A scalable laser patterning process may be utilized to produce highly conductive, stretchable electrodes capable of directly interfacing with human skin. The electrodes and sensing devices incorporating the electrodes may find applications in continuous health monitoring, human-machine interfaces, and wearable diagnostics, among other uses.

Unless otherwise specified, all technical and scientific terms used herein have the meanings commonly understood by persons skilled in the art to which this invention pertains. Any definitions provided herein are intended to supplement these common understandings and are not meant to be limiting.

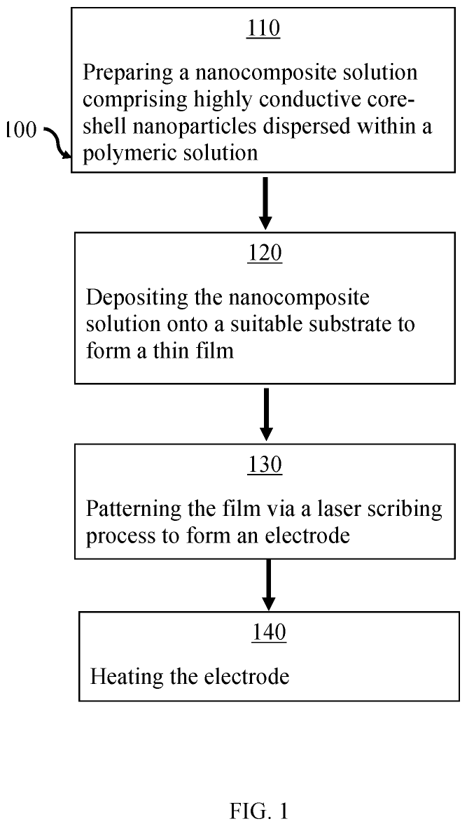

Referring to FIG. 1, an exemplary process 100 of forming an electrode includes preparing a nanocomposite solution (step 110). The nanocomposite solution includes highly conductive nanoparticles dispersed within a polymeric solution.

The nanoparticles may be formed from one or more metals selected from the group consisting of silver, gold, platinum, palladium, titanium, copper, zinc, and/or mixtures thereof.

The nanoparticles may be core-shell nanoparticles, which include a central core formed from a first metal or metal oxide selected from the list above, which is encapsulated by an outer shell formed from a second metal or metal oxide selected from the list above. In some embodiments, the first (core) metal or metal oxide may be silver. In some embodiments, the second (shell) metal or metal oxide may be copper.

The core-shell nanoparticles may advantageously include silver-copper core-shell nanoparticles. Such nanoparticles can be highly dispersible and demonstrate high conductivity.

The polymeric solution includes one or more polymeric components selected from the group consisting of polyvinyl alcohol (PVA), ethylene vinyl alcohol (EVOH), polyvinylpyrrolidone (PVP), polyethylene glycol (PEG), vinyl acetate ethylene (VAE), poly(acrylic acid) (PAA), polyacrylic acid sodium salt (PAAS), carboxymethyl cellulose (CMC), hydroxypropyl methylcellulose (HPMC), and/or mixtures thereof.

The polymeric solution may further include additional components. The solution can include one or more plasticizers, which can increase the flexibility and stretchability of the electrode. The solution can further include one or more humectants, which can help maintain moisture content within the polymer matrix, which can aid in the local deformation and conformal contact mechanism of the electrode. The solution can further include one or more lubricants. The solution can further include one or more reinforcing fillers to improve the mechanical strength, flexibility, and/or stability of the electrode.

In some embodiments, the solution can include one or more components that function as a plasticizer, lubricant, and/or humectant. Moreover, the presence of hydroxyl groups (—OH) in glycerol may enhance the hydrophilicity of the electrode to promote strong adhesion to human skin in the presence of water molecules. The component may be glycerol, propylene glycol, ethylene glycol, sorbitol, PEG, xylitol, mannitol, and/or mixtures thereof.

In some embodiments, the solution can include one or more components that function as a reinforcing filler. The components may be montmorillonite (MMT), kaolin, halloysite nanotubes, laponite, silica nanoparticles, and/or mixtures thereof.

In one embodiment, the polymeric solution may include a mixture of PVA, PEG, glycerol, and MMT.

The nanocomposite solution may include from 50 wt % to 90 wt % of nanoparticles, based on the total weight of the nanocomposite solution. The high conductivity of the electrode can be attributed to the use of highly concentrated conductive nanoparticles. However, less than 50 wt % of nanoparticles may not provide adequate conductivity. Moreover, greater than 90 wt % of nanoparticles may adversely affect the conformity of the electrode.

In nonlimiting embodiments, the nanocomposite solution may include no less than 50 wt %, no less than 55 wt %, no less than 60 wt %, no less than 65 wt %, no less than 70 wt %, no less than 75 wt %, no less than 80 wt %, no less than 85 wt %, and/or the like, of nanoparticles. In further nonlimiting embodiments, the nanocomposite solution may include no greater than 90 wt %, no greater than 85 wt %, no greater than 80 wt %, no greater than 75 wt %, no greater than 70 wt %, no greater than 65 wt %, no greater than 60 wt %, no greater than 55 wt %, and/or the like, of nanoparticles.

After the nanocomposite solution is prepared, the nanoparticles may coalesce at or near room temperature. This can result in a viscous, homogeneous solution suitable for film/electrode formation.

The nanocomposite solution may then be deposited onto a suitable substrate to produce a thin film (step 120).

The film may subsequently be patterned using a laser scribing process to form the electrode (step 130). In particular, a laser beam can selectively ablate regions of the film to define the desired sensor geometries. This approach enables the preparation of a variety of on-skin sensors with complex geometric properties, as will be described in further detail below.

After laser scribing, the patterned film can optionally be further processed (step 140), such as to enhance the electrical and/or mechanical properties of the resulting electrode. For example, the patterned film may undergo one of the following processing steps: thermal annealing, solvent vapor annealing, controlled drying, hot pressing, and/or cross-linking. Such steps can promote chain relaxation, improve component dispersion, and/or form more uniform, conductive, and compact films.

The resulting electrode may achieve high conformity with a user's skin. In particular, the conformal contact mechanism relies on the partial dissolution of one or more components of the polymeric solution (e.g., PVA and/or MMT), which can locally soften and deform in the presence of water. For example, water may be placed between a user's skin and the electrode prior to placing the electrode on the skin. The electrode, and specifically the water-responsive polymer matrix, may therefore create a flexible, deformable interface that can adapt to the shape and contour of the skin.

Due to the electrode's ability to achieve high conformal contact with the user's skin, the electrode can further achieve increased signal quality and reduced impedance relative to other materials. These properties can be maintained even during skin movements, stretching, and deformation.

In some embodiments, the electrode can maintain a sheet resistance at or below 1.2 Ω/sq upon stretching the electrode up to 10%, up to 20%, and even up to 30%.

The electrode may further achieve strong adhesion to the user's skin in the presence of water molecules. This can be the result of hydroxyl groups present in one or more components (e.g., PVA and/or MMT) of the polymeric solution.

However, the electrode may simultaneously exhibit an “easy to remove” feature when wetted with water. For example, as explained above, due to the water-deformable nature of the electrode, it can be easily removed by rinsing with water, leaving no residual adhesive or skin irritation. This water-responsiveness also permits easy cleaning and reapplication. This further allows the electrode to apply to all skin types, including delicate skin, such as the skin of an infant user or of an elderly user.

Ultimately, the electrode can simultaneously exhibit at least the following qualities: (i) high interfacial conformability, (ii) high electrical conductivity, and (iii) high stretchability. Overall, thanks to the flexible and stretchable properties of the electrode, it can deform with the skin during various mechanical deformations, leading to minimal changes in the effective contact area and interfacial gap distance. As a result, low contact impedance can be maintained to ensure high signal quality and low noise.

Embodiments further relate to a sensing device 300 incorporating the electrode 200 formed from the process described herein. The device 300 may encompass a variety of wearable, portable, and/or integrated systems designed for continuous health monitoring, human-machine interfaces (HMIs), and/or wearable diagnostics.

The device 300 may be configured for continuous and/or intermittent electrophysiological monitoring. In this configuration, the electrode 200 can be applied directly onto a user's skin at targeted locations to collect electrical signals generated by physiological activity. The device 300 may include signal conditioning circuitry, wireless transceivers, and power sources, enabling real-time data collection, processing, and transmission without impeding user mobility.

The data acquired by the device 300 may include various metrics, such as human motion, gestures, and physiological signals. These metrics can encompass motion trajectories, positional data, orientation, rotational movements, and other kinematic parameters, which can be obtained via the electrodes 200.

For example, in wearable health monitoring applications, the device 300 can be configured as an electrophysiological (EP) electrode, which may be used to record and analyze electrocardiograms (ECG), electromyograms (EMG), and electroencephalograms (EEG). These signals, which are generated during muscular, cardiac, ocular, or neural activity, can contain a wealth of physiological information reflective of the body's health status.

Furthermore, the collected electrophysiological data may be employed in HMI systems, where muscle activity signals (e.g., EMG) may be translated into control commands, such as for robotic limbs or assistive devices. The electrodes' capability to accurately capture subtle muscle or neural signals enables intuitive and responsive control interfaces, advancing the development of wearable electronics for immersive and natural user experiences.

The electrode 200 may be mounted on various parts of a user's body, including but not limited to, the chest, limbs, or face, depending on the targeted physiological signals.

The electrode 200 may alternatively be configured as a temperature sensor. For example, the electrode can serve as a sensor to collect the body temperature of a user. The electrode 200 can alternatively be configured as a humidity sensor. For example, the electrode can serve as a sensor to detect changes in moisture levels during respiration, skin perspiration, etc. The former can be used to estimate health conditions, while the latter allows the characterization of skin conditions.

The electrode 200 can alternatively be configured as a pulse oximeter, which can measure pulse rate, blood oxygen saturation, etc.

The electrode 200 can also be used as an interconnect or circuit board that is integrated with commercial off-the-shelf chips.

A device 300 can include one or a plurality of electrodes 200. For example, a device 300 may include multiple electrodes 200 operating independently or collectively. Each electrode 200 can function without interference, enabling distributed sensing or energy harvesting across different body parts. The number of electrodes 200 can range from one to many, including configurations with two, three, four, or more.

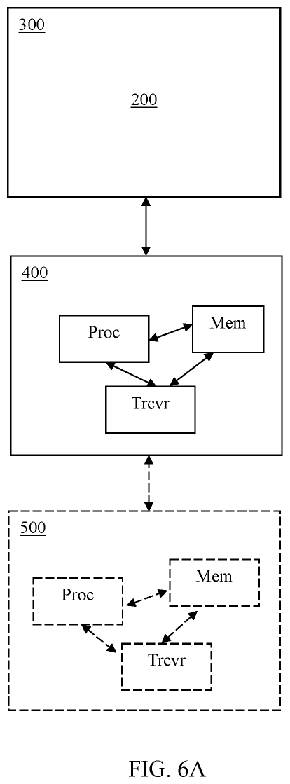

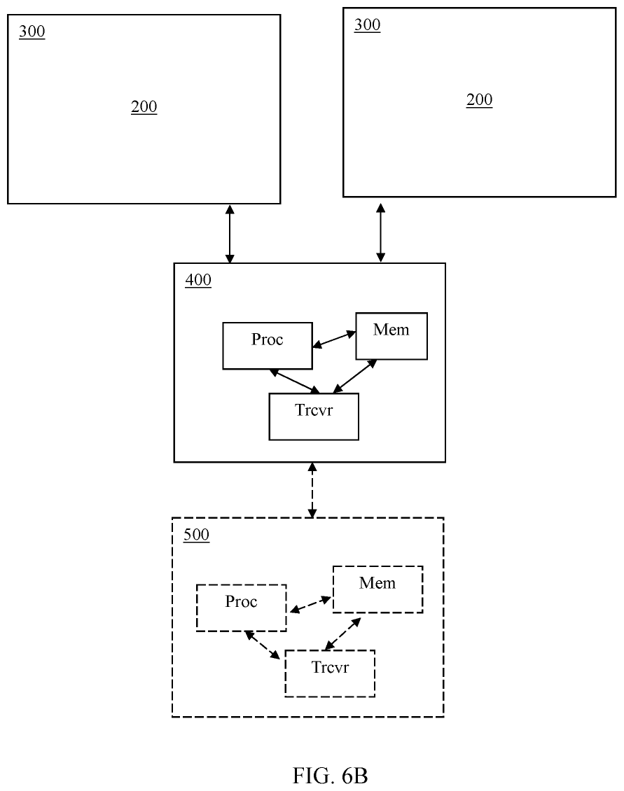

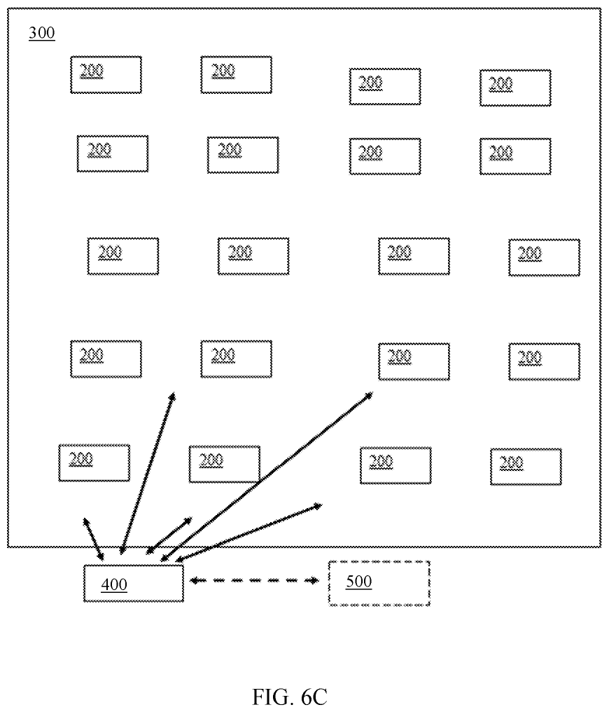

Referring to FIGS. 6A, 6B, and 6C, the device 300 can be hardwire connected to an input/output device 400 (e.g., a smart phone, tablet, laptop computer, personal computer, computer device of a drone or robotic device, etc.), or the device 300 can be communicatively connected to the input/output device 400 via a network connection or wireless connection (e.g., internet connection, wide area network connection, near field communication connection, Bluetooth connection, etc.).

In some embodiments, the input/output device 400 can be configured to receive data from the device 300 for storage and analysis. In some implementations, the input/output device 400 can be configured as a server or cloud-based service providing device for storage and analysis of the data obtained via the device 300. The data can be communicated to a user via a display device, which can be a tablet, smart phone, laptop computer, personal computer, or other type of terminal device. The display device can be effectuated via an application programming interface (API) and/or the use of an application stored on the display device. It is contemplated that the input/output device 400 can comprise the display device, or the display device can be a separate device.

In some embodiments, the device 300 can alternatively or subsequently be sent to a central computer device 500 (e.g., a server, an operator workstation, etc.) that can be hardwire connected to the device 300 and/or the input/output device 400, or can be communicatively connected to the device 300 and/or the input/output device 400 via a network connection and/or a wireless connection. The central computer device 500 can be configured to store, analyze, and/or display data received from the device 300 and/or the input/output device 400.

In some embodiments, the collected data can be continuously streamed to the input/output device 400 and/or the central computer device 500. In other embodiments, the collected data can be periodically streamed to the input/output device 400 and/or the central computer device 500 (e.g., non-continuously at pre-determined intervals). Embodiments of the device 300 can be configured to provide real-time data collection.

The input/output device 400 and/or the central computer device 500 can be a computer device that can include a processor (Proc.) connected to a non-transitory memory (Mem.) and at least one transceiver (Trcvr) for forming communicative connections with one or more other devices. The at least one transceiver (Trcvr) can include a Bluetooth module and/or other type of transceiver unit (Trcvr). The processor can be hardware (e.g., processor, integrated circuit, central processing unit, microprocessor, core processor, computer device, etc.), configured to perform operations by execution of instructions embodied in algorithms, data processing program logic, artificial intelligence programming, automated reasoning programming, etc. that can be defined by code stored in the memory. The processor can facilitate receipt, processing, and/or storage of readings from the v and/or control transmission of the collected data to the input/output device 400 and/or the central computer device 500.

It should be noted that use of processors herein can include hardware, such as for example any one or combination of a Graphics Processing Unit (GPU), a Field Programmable Gate Array (FPGA), a Central Processing Unit (CPU), a microprocessor, a processor, etc. The processor can include one or more processing or operating modules. A processing or operating module can be a software or firmware operating module configured to implement any of the functions disclosed herein. The processing or operating module can be embodied as software and stored in non-transitory memory, the memory being operatively associated with the processor. A processing module can be embodied running a web application, a desktop application, a console application, etc.

The memory (Mem.) can be a non-transitory computer-readable memory configured to store data. Embodiments of the memory can include a processor module and other circuitry to allow for the transfer of data to and from the memory, which can include to and from other components of a communication system. This transfer can be via hardwired links or wireless transmission communication links. The communication system can include transceivers, which can be used in combination with switches, receivers, transmitters, routers, gateways, waveguides, etc. to facilitate communications between different devices via a communication approach or protocol for controlled and coordinated signal transmission and processing to any other component or combination of components of the communication system. The transmission can be via a communication link, which can be a wireless type of communication connection and/or a wired type of connection.

The computer or non-transitory machine-readable medium can be configured to store one or more instructions thereon. The instructions can be in the form of algorithms, program logic, etc. that cause the processor to execute any of the functions disclosed herein.

The processor can be in communication with other processors of other devices (e.g., additional external devices, a computer system, a laptop computer, a desktop computer, etc.). An exemplary other device can be a Bluetooth-enabled device, near field communication device, etc. Any of those other devices can include any of the exemplary processors disclosed herein as well as transceivers or other communication devices/circuitry to facilitate transmission and reception of wireless signals or other types of communicative connections.

Either the input/output device 400 and/or the central computer device 500 can be configured to be connected to other input devices and output devices. Examples of input devices can include a scanner device (e.g., scanner), a microphone, a keyboard, a touch screen, a button, a sensor, a detector, or other type of input device. Examples of output devices can include a display, a printer, a speaker, or any other type of output device.

As noted above, once collected data is transmitted to the input/output device 400 and/or the central computer device 500, the data can be analyzed and evaluated to determine an output. For example, the data can be processed and evaluated to determine motion or gestures, to detect conditions or diseases, etc. In some embodiments, the data can be processed using artificial intelligence or machine learning algorithms stored on the input/output device 400 and/or the server central computer device 500. In particular, the input/output device 400 and/or the server central computer device 500 can run a program that uses the collected data along with a module trained via a machine learning process that received the collected sensor data and processes that data to determine an output.

### EXAMPLES

### Experimental Section

Materials: The poly(vinyl alcohol) (PVA-124) was purchased from Innochem (Beijing, China). The poly(ethylene glycerol) and montmorillonite (K-10) were obtained from Aladdin (Shanghai, China). The glycerol was purchased from Sinopharm Chemical Reagent Co. (Shanghai, China). The defoamer (BYK-024) was acquired from BYK (Wesel, Germany). The Ag—Cu nanoparticles were obtained from Ha Shen Technology Co. (Shenzhen, China).

Fabrication of the Skin-Conformal Membrane: The fabrication of the skin-conformal membrane started with mixing 9 wt % PVA, 5 wt % PEG, and 4 wt % glycerol in 81 wt % water, followed by heating at 90° C. After adding 1 wt % ST 2436 defoamer to eliminate bubbles, 13 wt % montmorillonite and 66 wt % Ag—Cu NPs were introduced to the 21 wt % obtained solution. Next, the mixture was deposited on a glass slide with a doctor's blade to achieve a uniform membrane. Finally, the membrane was heated to 50° C. for half an hour to achieve enhanced electrical conductivity.

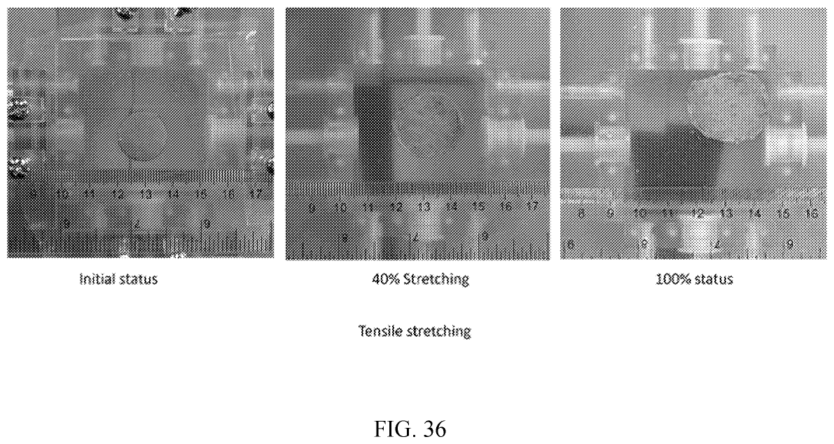

Tensile Test of the Membrane: The membrane was first attached to a soft 00-30 Ecoflex™ substrate with a size of 2 cm×2 cm×5 mm, and then a small amount of water was applied at the interface to improve the adhesion. After clamping both ends of the composite membrane, a custom-built stretcher was used to apply uniaxial tensile strain and the electrical resistance was simultaneously measured with the Keithley 2401 digital multimeter (FIG. 36).

Fabrication and Characterizations of the Sensors: The different types of sensors with various 2D patterns were designed by AutoCAD and patterned with a CO₂ laser-cutting machine (Universal Laser System, ULS 2.3). The patterned sensors were connected to conductive copper/polyimide (DuPont Pyralux AC Single-side Clad, Copper/Dielectric thickness: 09 μm/12 μm) thin stripes via conductive silver paste (MG Chemicals, 8331D). The electrical resistance of the sensors was measured by connecting the copper stripes to a Keithley 2401 digital multimeter (with data acquisition I-V software) with alligator clips. The sheet resistance Rₛ was then calculated from the electrical resistance R as Rₛ=R·L/W, where L and W are the length and width of the samples. The capacitance of the humidity sensor was measured by an LCR meter (Hioki IM 3536 01). The subject inserted the arm with the humidity sensor into a sealing jar together with a cup of water. The evaporation of the water will raise the moisture level in the jar and therefore change the capacitance of the humidity sensor. The power of the heater was provided by a DC power supply (EVENTEK, KPS3010d), and the temperature was captured by the FLIR infrared camera. The impedance was measured by KEYSIGHT E4980A. Electrophysiological signals (e.g., ECG, EMG, and EEG) were collected by PowerLab with Bio Amp (ADInstruments). The Fourier transform was obtained by MATLAB using the FFT function. All experiments on human subjects were approved by the Institutional Review Board (IRB) at the Pennsylvania State University (STUDY00008003).

Measurement and Comparison of Surface Morphology: A PDMS elastomer finger mold was first prepared by curing the precursor (SYLGARD 184, 10:1 ratio, Dow Corning) against the thumb finger. Casting the liquid wax into the PDMS finger mold prepared the wax-based finger mold. Pressing the membrane against the wax-based finger mold for 10 seconds created the fingerprint on the thin film. The flat PMDS (SYLGARD 184, 10:1 ratio, Dow Corning) substrate with the spiral pattern designed by AutoCAD was created by the CO2 laser system (Universal laser system VLS2.30) with specified parameters (power 10.5%, speed 10 mm/s, PPI 1000). After finger pressing the membrane against the patterned PDMS substrate for 10 seconds, drying the membrane in the ambient environment was followed by removal from the PDMS surface. The surface morphology of the dried membrane and patterned PDMS substrate was then measured with an optical profilometry device (Zygo NexView3D).

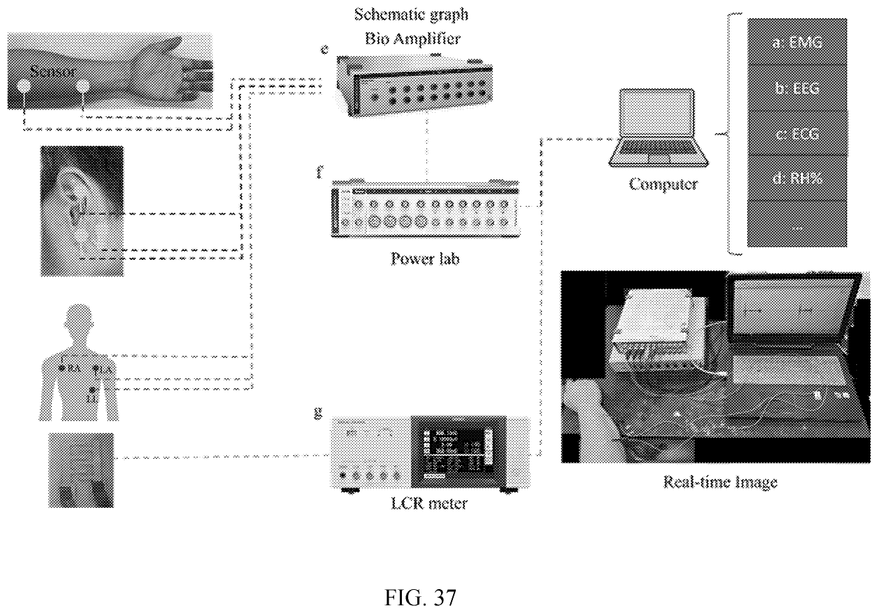

Three-Lead ECG Measurement: ECG signals were collected from three-lead electrodes using Power Lab with Bio Amps from AD Instruments (FIG. 37). The fabricated electrodes were placed on the right arm (RA), left arm (LA), and left leg (LL) of the subject. Then, the electrodes were connected to the common, positive, and negative electrode portals of the Bio Amps. The Bio Amps were then used to transfer the signal to the Power Lab, and the signals were read from the computer connected to the Power Lab. The potentials obtained by the data acquisition system were generated by the contraction of the cardiac muscles in the cardiovascular cycles.

Tripolar Electrodes for EEG Measurement: The fabricated electrodes were placed on the following locations of a subject: scalp, around the ears, and inside the ear (by inserting an earplug with an electrode attached to it). Then, the electrodes were connected to the Bio Amps as stated in the “Three-Lead ECG Measurement” description above. The potential obtained by the data acquisition system was generated from the spontaneous electrical activities of the brain. Then, the data was under the following detailed derivation: the Laplacian of the potential ΔP from the tripolar concentric ring electrode was approximately calculated as: ΔP≅16(Vₘ−V_{c})−(V_{O}−V_{c}), where V_{c}, Vₘ, and V_{O} are the potentials on the center, middle, and outer rings, respectively.

EMG Measurement: The fabricated electrodes were placed on the same muscle of the subject, with a reasonable distance between the two electrodes. Then, the electrodes were connected to the positive and negative electrode portals of Bio Amps. Other connections were the same as stated in the “Three-Lead ECG Measurement” description above. The potential obtained by the data acquisition system was generated from the contraction of the muscle.

Human-Machine Interface Device Setup: EMG signals measured by Power Lab were transferred to MATLAB for real-time game control. The combined EMG signals obtained from the left and right inner forearm muscles were used to generate four control commands: the extension of the left (or right) forearm with contraction of the right (or left) forearm for “left turn” (or “Right turn”), the extension of both for “move forward”, and the contraction of both for “move backward”. The Support Vector Machine (SVM) algorithm was then used to process the EMG signal peak magnitude for command classification with an accuracy of 95.5%.

### Results

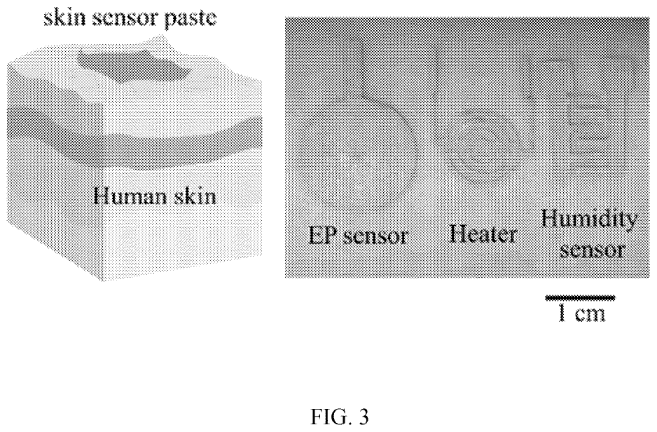

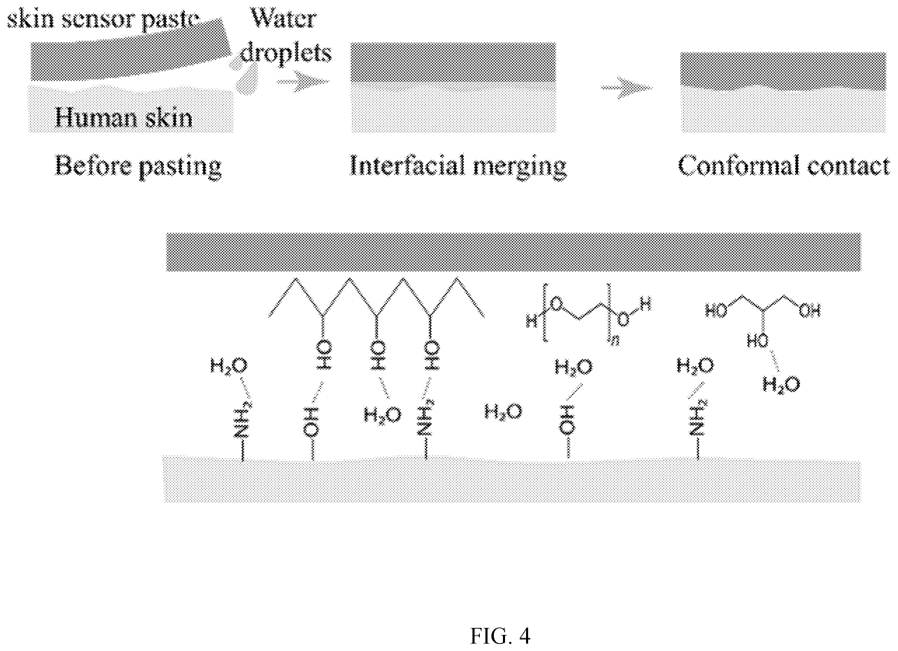

Material Performance: The nanocomposite thin film features exceptional interfacial conformability, high electrical conductivity, and good stretchability. Facilitated by interlayer water molecules, the thin film forms conformal contact with the skin (FIG. 3). By exploiting a laser patterning process, various high-performance skin-interfaced sensors can be facilely fabricated, including EP, humidity, temperature sensors, and heaters. The excellent interfacial conformability results from partial dissolution and local deformation of MMT and PVA (FIG. 4). Furthermore, the massive presence of hydroxyl group (—OH) in PVA and glycerol enhances the hydrophilicity to result in strong adhesion to human skin in the presence of water molecules.

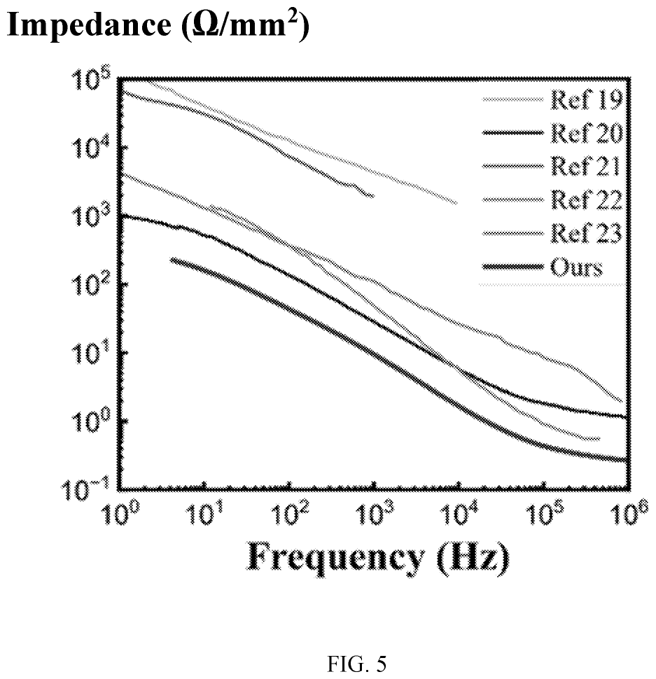

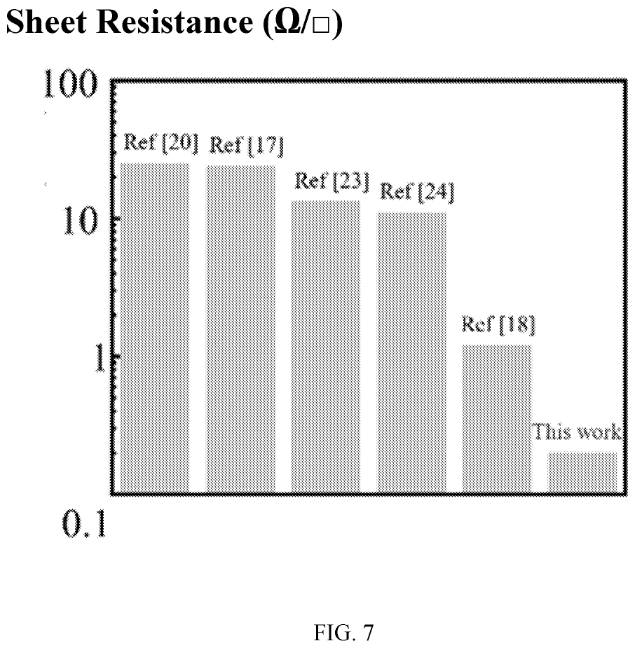

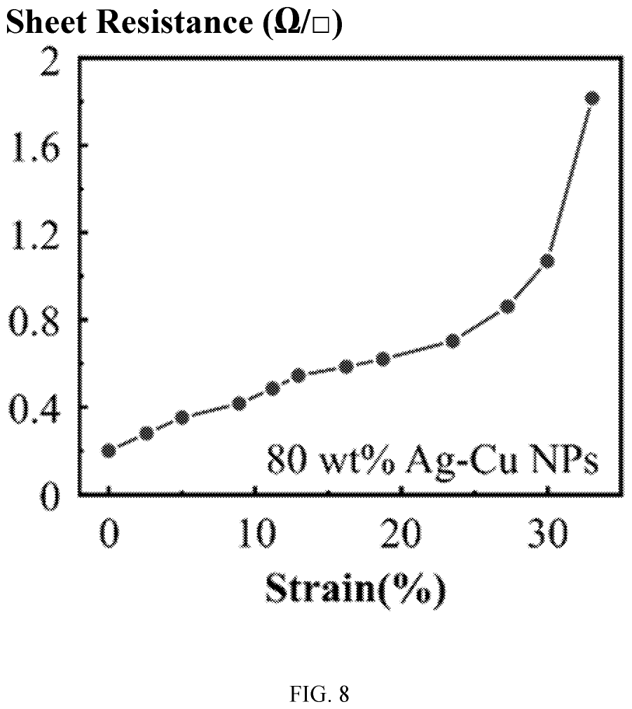

The excellent contact at the electrode/skin interface results in low areal contact impedance over a wide range of frequencies (FIG. 5), which is significantly lower than those previously reported based on flexible materials. In addition, the thin film exhibits excellent electrical conductivity with reduced sheet resistance compared to the previous reports based on flexible materials (FIG. 7). The high conductivity can also be well maintained upon stretching of >30% (FIG. 8), which is larger than the maximum strain on the skin. The outstanding conformal contact, high electrical conductivity, and stretchability are ideal for motion artifact-free sensing and on-skin bioelectronics.

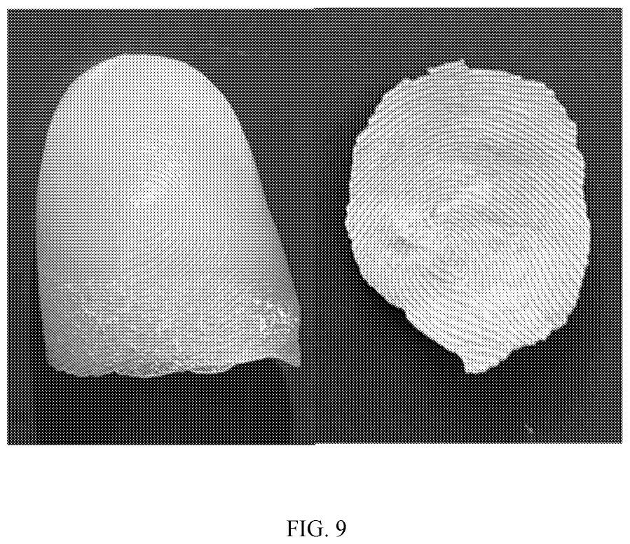

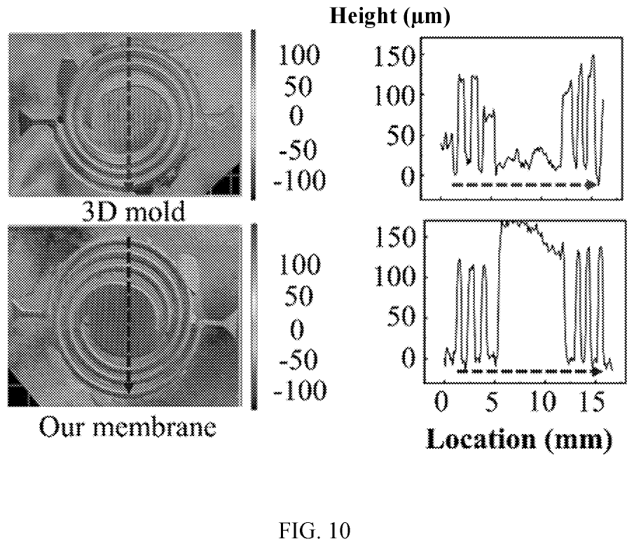

Partial dissolution and local deformation allow the thin film to conform to the skin. The conformal contact is quantified by comparing the 3D morphology of the wax mold of thumb-fingerprint patterns and the conformed thin film (FIG. 9). After partial dissolution and local deformation, the thin film conforms to the wax mold with hierarchical fingerprint structures. The morphologies of the 3D wax mold and the conformal thin film captured by the optical profilometer both show distinct peaks and valleys on the surface (FIG. 10). The average heights between peaks and valleys exhibit a high degree of similarity for the 3D max mold (119.79 μm) and thin film (135.13 μm), suggesting an excellent match of 88.65% in the interfacial morphology.

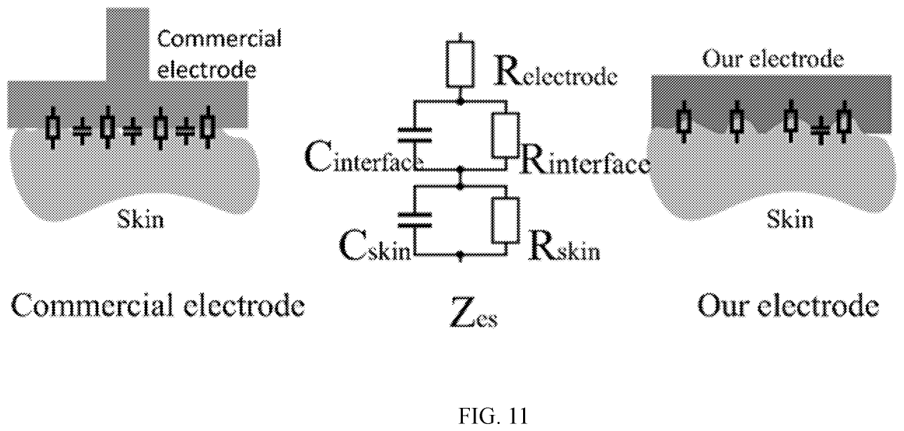

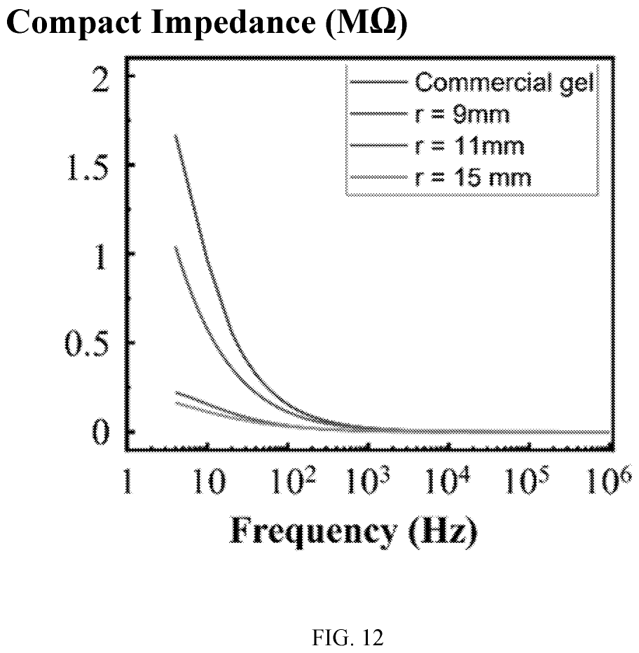

The conformal contact contributes to the measured low contact impedance at the electrode/skin interface. The electrode-skin impedance (Zₑₛ) is composed of several components, including the electrode resistance (R_{electrode}), the electrode-skin contact resistance (R_{interface}), the electrode-air-skin capacitance (C_{interface}), and the skin impedance (Rₛₖᵢₙ∥Cₛₖᵢₙ) (FIG. 11). The amplitude of the overall impedance (Zₜₒₜₐₗ) between two electrodes is the sum of two electrode-skin impedances (Zₑₛ₁ and Zₑₛ₂) and the bioimpedance of the human body (Z_{bio}): |Zₜₒₜₐₗ|=|Zₑₛ₁+Zₑₛ₂+Z_{bio}|. The highly conductive electrode (and commercial gel electrode) exhibits a small electrode resistance (R_{electrode}), which has a negligible effect on the total impedance. Because the skin impedance (Rₛₖᵢₙ∥Cₛₖᵢₙ) and body impedance (Z_{bio}) remain unchanged for the comparison experiment between our and commercial gel electrodes on the same human subject, the total impedance is mostly determined by the electrode-skin contact resistance (R_{interface}) and the electrode-air-skin capacitance (C_{interface}). The partial dissolution and local deformation of the thin film allow it to partially fill the air gap between the electrode and the hierarchical skin, increasing the contact area (A) and decreasing the air gap distance (d). As a result, the electrode-skin contact resistance (R_{interface}) is reduced and the electrode-air-skin capacitance (C_{interface}) is increased, leading to the decreased overall impedance noting that |Z|=[(1/R)²+(ωC)²]^{-1/2}. The increased size of the electrodes also decreases the contact impedance (FIG. 12).

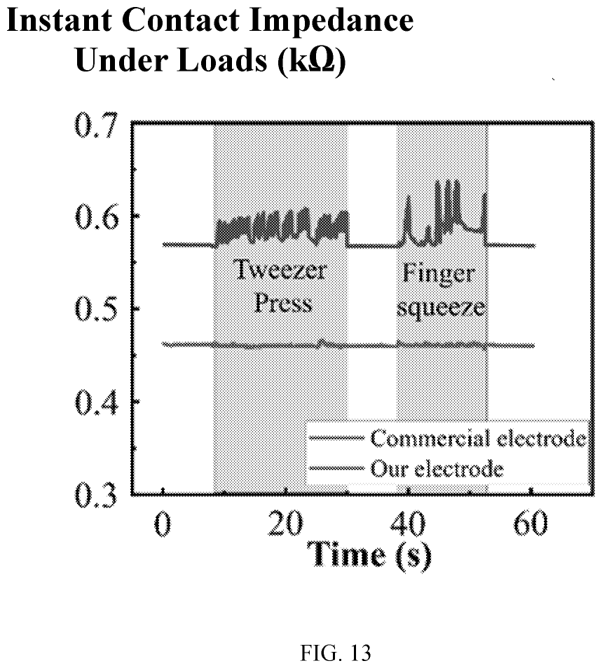

Thanks to the flexible and stretchable properties, the thin film deforms with the skin during various mechanical deformations, leading to minimal changes in the effective contact area and interfacial gap distance. As a result, the low contact impedance can be maintained to ensure high signal quality and low noise. For instance, the skin-electrode contact impedance at 100 k Hz remains at a consistently low value under external interference from the tweezer pressing and finger squeezing (FIG. 13). In contrast, the commercial gel electrode shows a large fluctuation in the impedance during external perturbation.

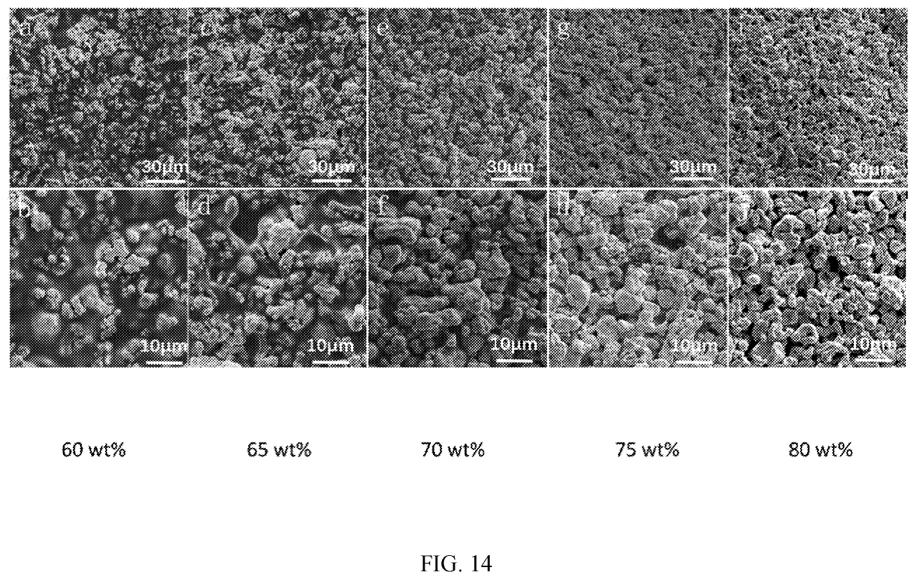

The high conductivity of the thin film can be attributed to the use of highly concentrated conductive Ag/Cu@Cu NPs. As the concentration of Ag/Cu@Cu NPs increases from 60 wt % to 80 wt % with a step size of 5 wt % (i.e., 60, 65, 70, 75, and 80 wt %), the contact of the metal NPs at the micrometer scale also increases as observed in the SEM image (FIG. 14). As a result, the sheet resistance is significantly reduced (FIG. 14).

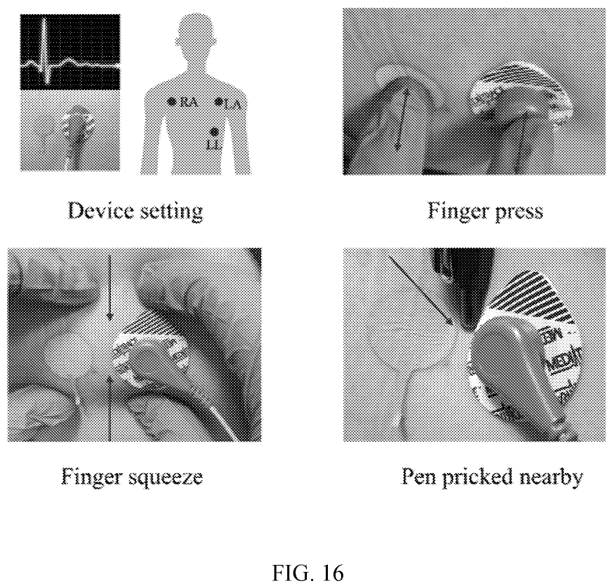

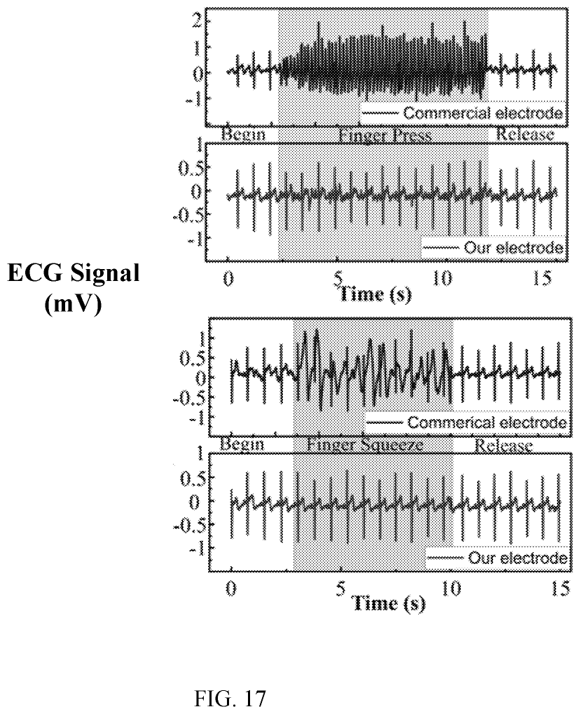

Motion Artifact-Free EP Sensing with Electrodes: The ultra-conformality and low contact impedance over deformation allow the resulting electrodes for motion artifact-free sensing of EP signals. As a representative example, the ECG signals collected by the three-lead conformal stretchable electrodes (FIG. 16) are not affected by the various external perturbations such as finger pressing and squeezing, whereas those from the commercial gel electrodes show significant fluctuations (FIGS. 17-19). The signal-to-noise ratio (SNR), which measures the strength of a signal relative to the background noise, is used to evaluate the performance of electrodes in the time domain. The value of SNR can be determined from the following equation: SNR=20 log(Vₛ/Vₙ), where Vₛ represents the peak-to-peak amplitude of the signal (the difference between the R and S peaks of the PQRST cycle in the ECG signal) and Vₙ corresponds to the peak-to-peak amplitude of the noise. The SNR of the ECG signals from the conformal stretchable electrodes only exhibits a slight decrease from 20.24 to 17.51 as the finger press is applied (FIG. 17 (top)). In comparison, the commercial gel electrodes demonstrate a smaller SNR of 18.02 before the deformation and further fail to capture clear ECG signals under deformation, which is likely attributed to significant deformation near the rigid plastic shell.

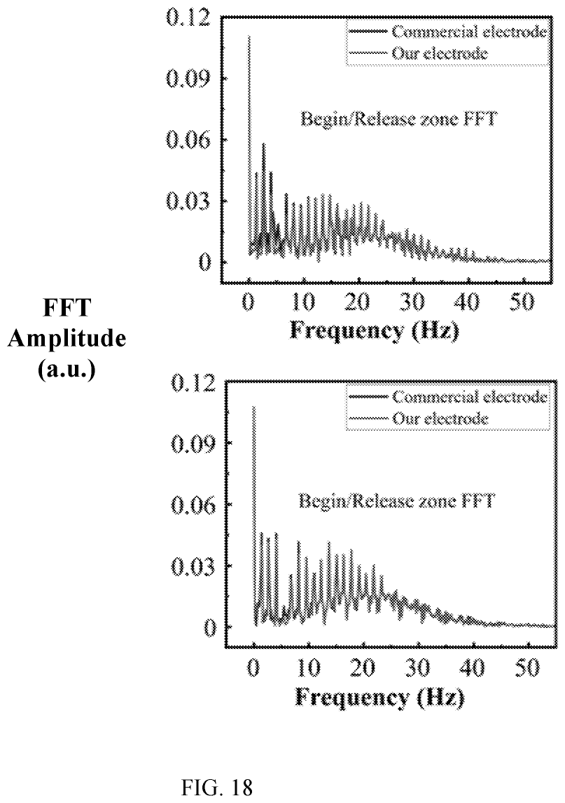

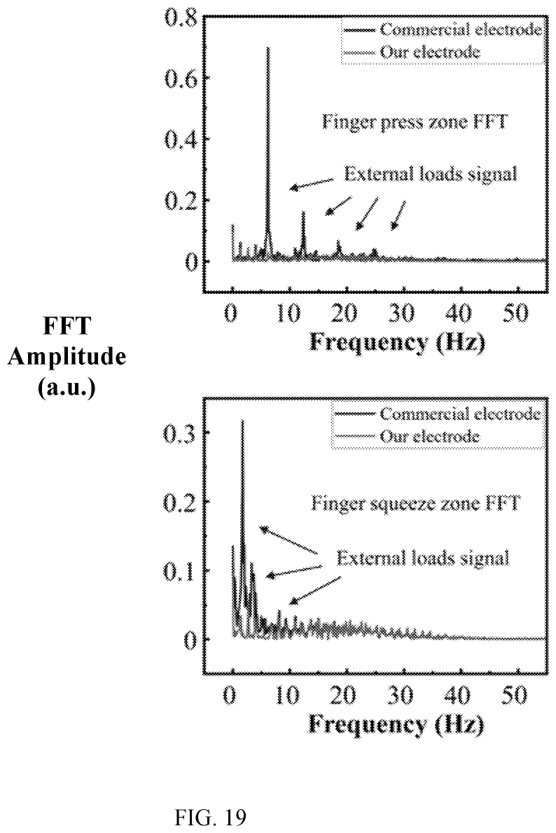

Transforming the ECG data from the time to frequency domain using the Fourier transform further reveals the impact of external mechanical deformations on the captured signals. Without external deformation, a high correlation in the Fourier-transformed ECG data between the conformal stretchable and commercial gel electrodes is observed to validate the accuracy of the conformal stretchable electrode (FIG. 18 (top)). As the finger pressing is applied, several sharp spikes with the frequency corresponding to the external loads show up in the signals captured by the commercial gel electrodes (FIG. 19 (top)). In contrast, no significant changes are observed in the signals captured by the conformal stretchable electrode, indicating motion artifact-free sensing of the EP signals with high accuracy and reliability. The impact of finger squeezing on ECG signals is similar to that of finger pressing in both the time domain and frequency domains (FIGS. 17-19 (bottom)).

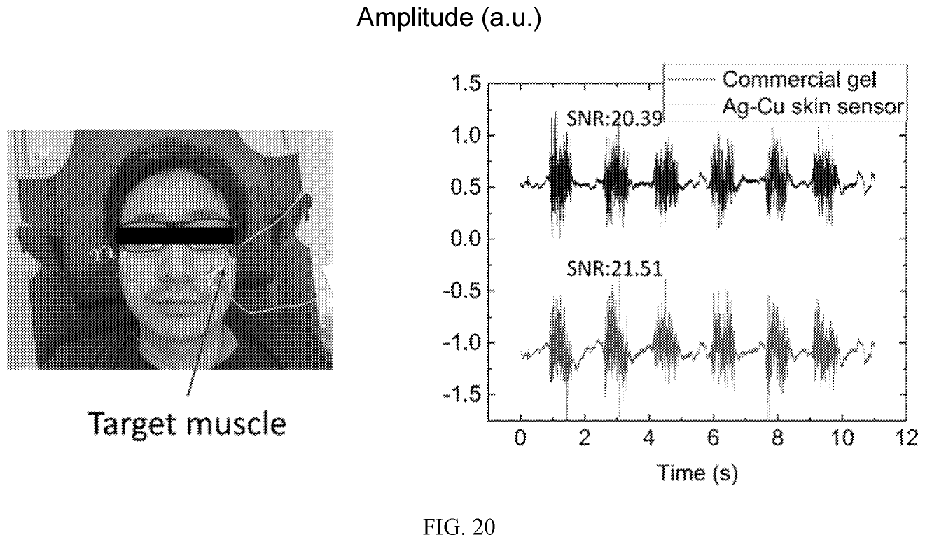

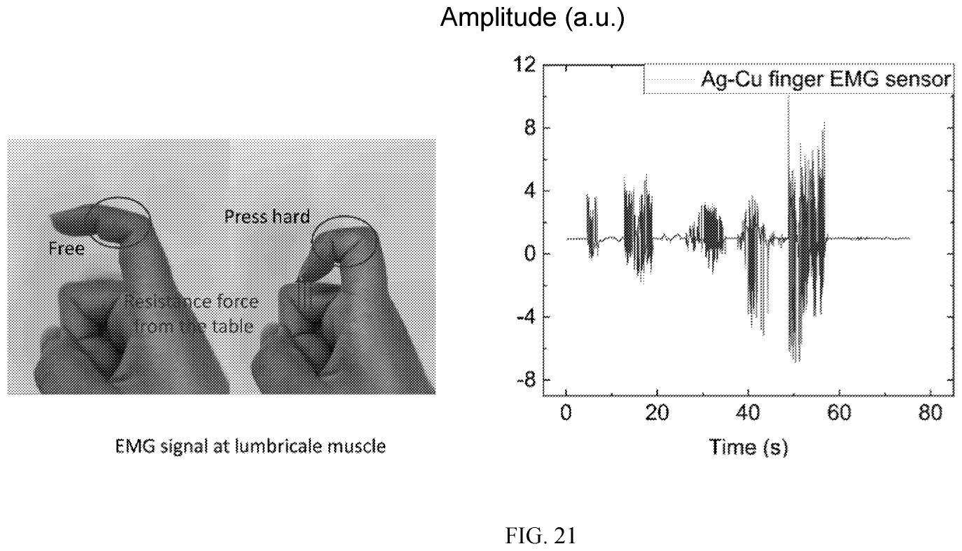

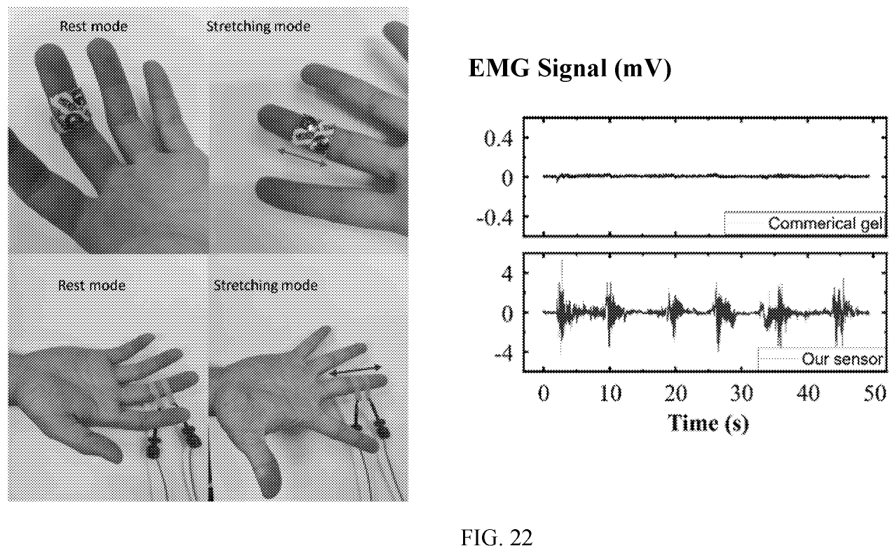

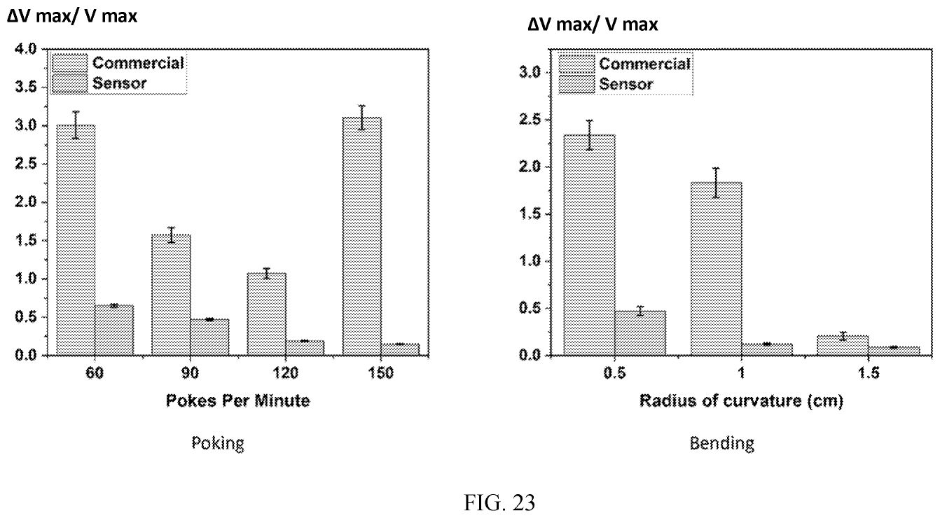

Similarly, the conformal stretchable electrodes can measure EMG signals from curvilinear surfaces such as facial muscles (FIG. 20) and finger muscles (FIG. 21) even in extension mode (FIG. 22), whereas the commercial gel electrodes fail to do so. In addition, we evaluate the deformation of electrodes with external loads and our sensors show less deformation compared with commercial electrodes (FIG. 23).

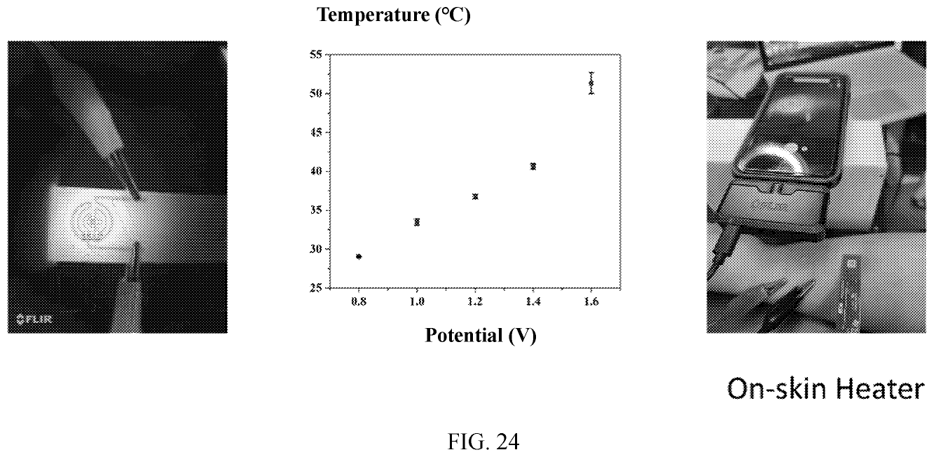

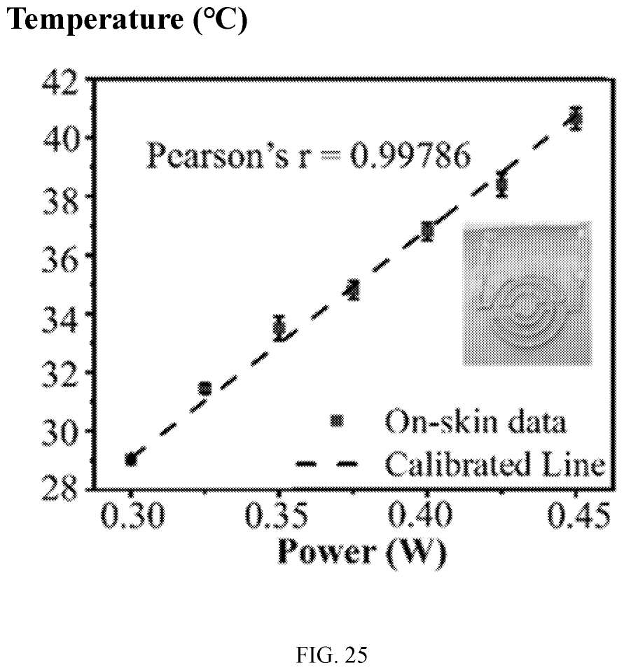

Demonstration and Application of Various On-skin Bioelectronics: The laser patterning approach can also prepare a variety of on-skin sensors with complex geometric patterns such as heaters/temperature sensors, humidity sensors, pulse oximeters, and in-ear EEG sensors for health monitoring, as well as a human-machine interface for game control. As flexible resistive heaters based on Joule heating are important for thermotherapy, an on-skin, spiral-shaped heater controlled by the input power is calibrated against a commercial thermometer (FIG. 24) and demonstrated for efficient heating (FIG. 25) toward wound healing and thermal management. The output peak temperature exhibits a strong linear relationship with the input power, with Pearson's linear correlation coefficient of 0.9979. Compared with previously reported soft heaters (Table 1), our spiral-shaped heater exhibits low sheet resistance, low drive voltage, and high heating temperature.

TABLE 1
Performance comparison between the heater from
this application (bottom row) and others.

| | Rs | Area | Temperature (° C.)/Applied |
|---|---|---|---|
| Heater Material | (Ω/sq) | (cm²) | Voltage (V) |
| Graphene Oxide | 1568 | 2 × 1.4 | 150/60 |
| Ag NW | 30 | 1.5 × 1 | 150/10 |
| Metallic glass | 3.8 | 2 × 2 | 120/5 |
| cupronickel | 16.2 | 2.5 × 2.5 | 135/6 |
| Ag/Graphene | 4 | 5 × 5 | 135/4 |
| Ag/Cu/MMT/PEG | 0.2 | 2 × 2 | 140/1.2 |

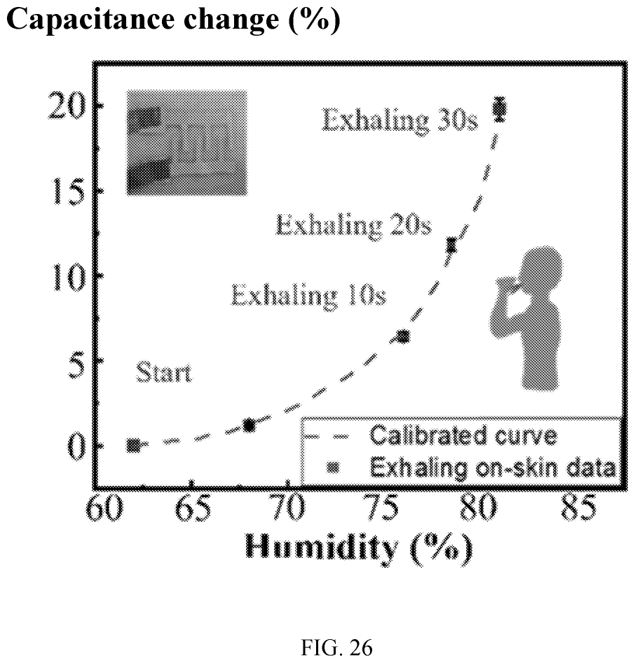

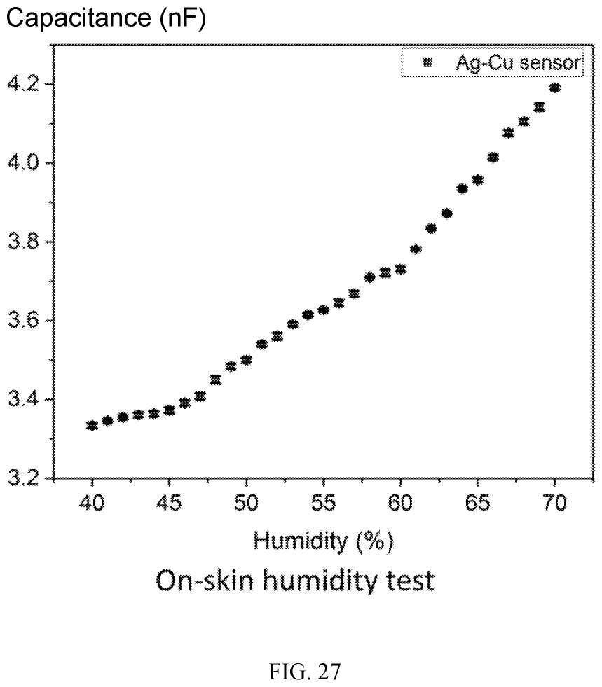

The humidity sensor is also very important to detect changes in moisture levels during respiration and skin perspiration. The former can be used to estimate health conditions, whereas the latter allows the characterization of skin conditions and barrier functions. Designed in an interdigital electrode (FIG. 26), the on-skin humidity sensor can detect the changes in humidity levels on the skin surface through the measured capacitance. After the calibration with a commercial humidity sensor (FIG. 27), humidity changes on the skin surface can be successfully measured during hydrous exhaling for various durations. The sensitivity of the humidity sensor is obtained as the ratio of the measured capacitance difference to the relative humidity difference, i.e., sensitivity=(C_{final}−C_{ref})/(RH_{final}−RH_{ref}).

The sensitivity of 1.913 nF/% RH in the range of 30%-90% from our humidity sensor is comparable to the previously reported values (Table 2).

TABLE 2
Performance comparison between capacitive humidity sensors.

| Sensitivity | Material | Humidity |
|---|---|---|
| 2.8 pF/% RH | Zinc Oxide | 40-90% RH |
| 0.85 pF/% RH | PMDA-ODA-TiO2 | 10-90% RH |
| 4 pF/% RH | Al2O3 | 5-85% RH |
| 8.2 pF/% RH | CaCl2 | 30-95% RH |
| 1.8 nF/% RH | PIL | 10-80% RH |
| 1.6 nF/% RH | Zn2SiO4 | 11-95% RH |
| 1.9 nF/% RH | Ag/Cu/MMT/PEG/Glycerol | 30-90% RH |

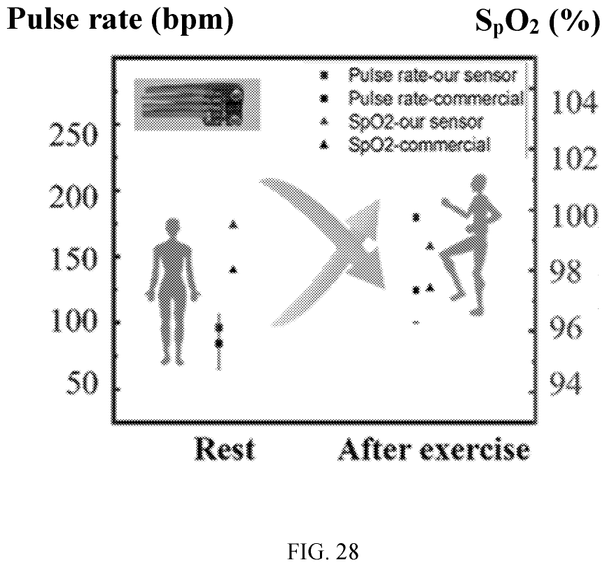

The highly conductive property of the conformal stretchable material can be used as interconnects to fabricate the flexible and stretchable printed circuit board. The integration of multifunctional sensors with commercial off-the-shelf chips allows for enhanced data processing/transmission capabilities to provide continuous health monitoring and early diagnosis. In a proof-of-the-concept demonstration, connecting the MAX 30100 sensing chip with an Arduino UNO microcontroller yields an integrated pulse oximeter (FIG. 28). The measured changes in pulse rate and blood oxygen saturation (SpO₂) levels before and after a 2-minute exercise agree reasonably well with those obtained from the commercial Fingertip Pulse Oximeter (Santa Medical).

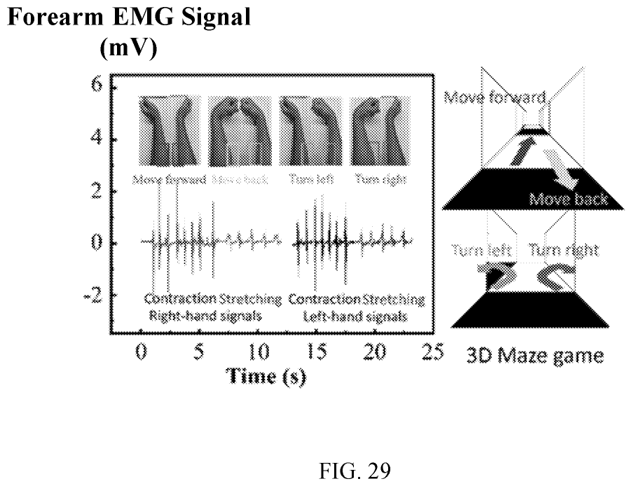

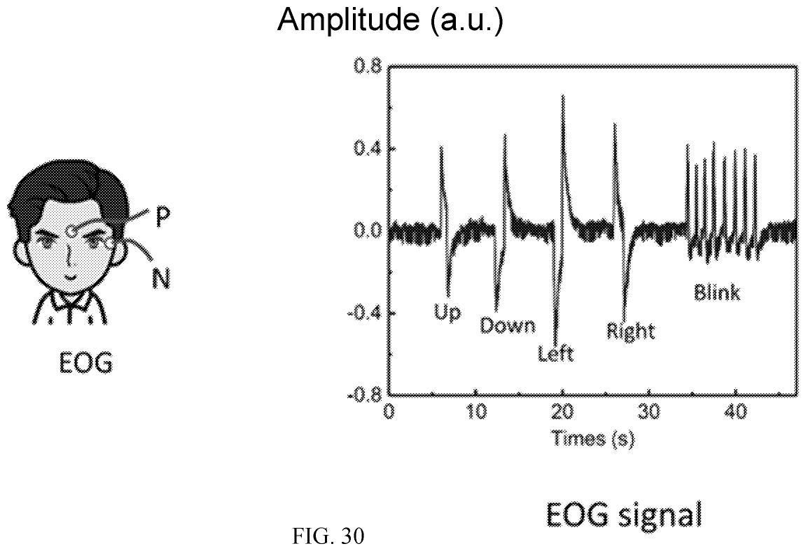

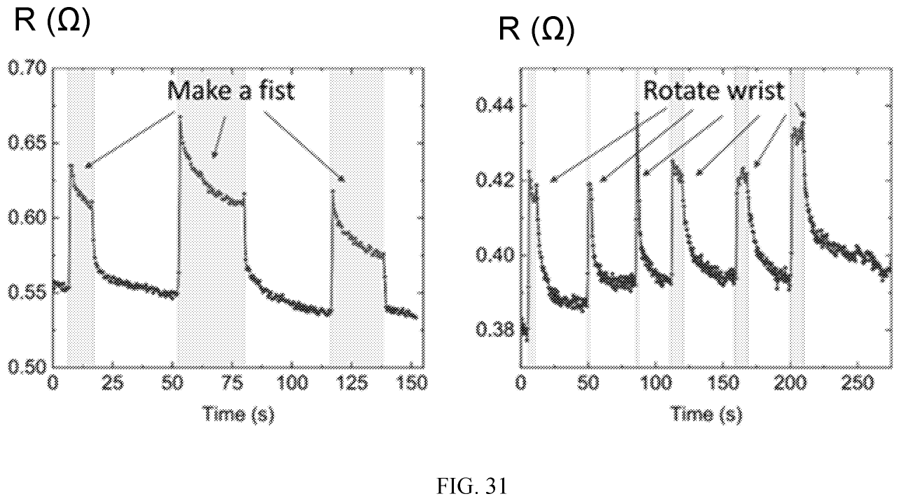

By facilitating efficient and effective communication between humans and machines, the human-machine interface combined with artificial intelligence can provide intuitive and user-friendly means to help humans interact with and manage complex systems. As a first step toward such a target, the acquired EMG signals from the inner side of both forearms are used to control a 3D maze game programmed in MATLAB in real-time (FIG. 29). By bending the wrist inward or outward (muscle contraction or stretching), distinct peak amplitudes in the EMG signals generate four gestures/commands to control the virtual person for moving forward, back, left, and right in the 3D maze game. With each gesture repeated 110 times, the demonstration achieves a high accuracy of more than 95.5% in gesture recognition with the human-machine interface (Table 3), demonstrating high robustness and reliability. Further demonstrations of gesture recognition include eye movements (FIG. 30) and hand gestures (FIG. 31).

TABLE 3
Accuracy table for gesture recognition.

| | Predict | Predict | Predict | Predict | |
|---|---|---|---|---|---|
| | forward | backward | left | right | Accuracy |
| Actual | 109 | 0 | 1 | 0 | 99.1% |
| forward | | | | | |
| Actual | 0 | 107 | 2 | 1 | 97.3% |
| backward | | | | | |
| Actual left | 4 | 0 | 106 | 0 | 96.4% |
| Actual right | 5 | 0 | 0 | 105 | 95.5% |

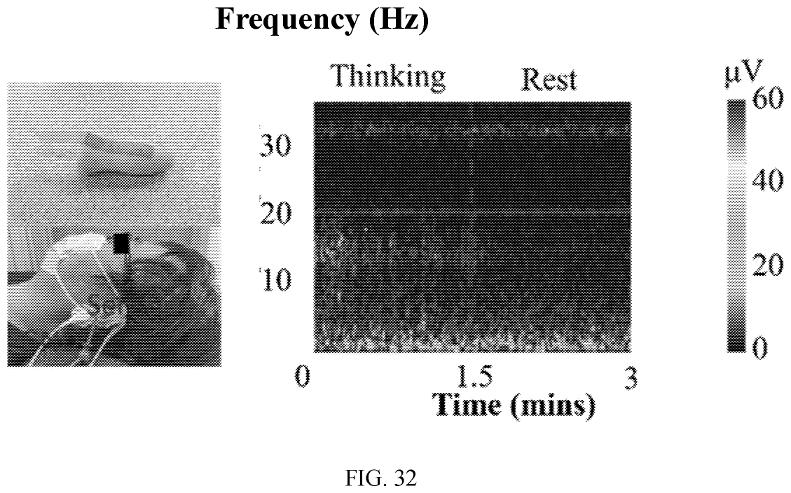

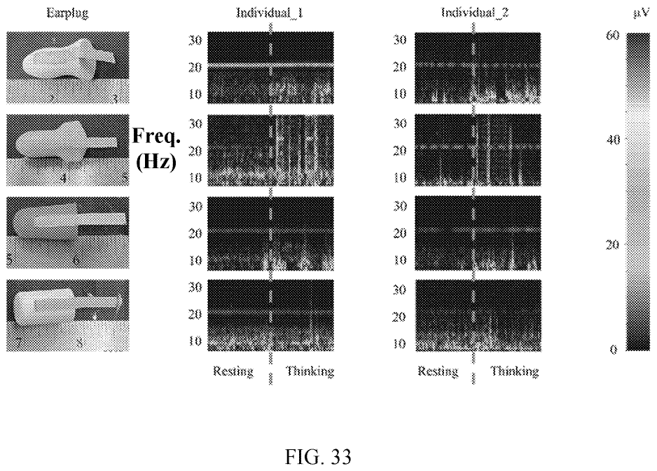

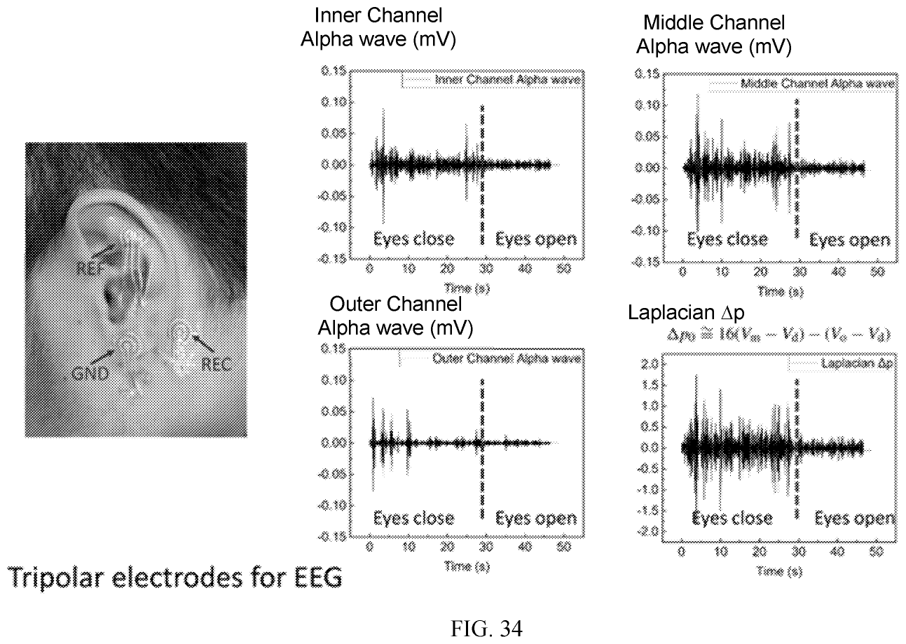

An accurate sleep study for cognitive evaluation relies on the measurement of EEG signals for an extended duration. Further analysis of sleep EEG signals can help reveal sleep patterns and brain activities, such as identifying biomarkers in depression, tracking rapid eye movement, and understanding anesthetic sedation. An in-ear EEG sensor that combines stretchable electrodes with commercial deformable foam earplugs is further designed to reduce interference during sleep (FIG. 32). The feasibility of the in-ear EEG sensor is first confirmed in a short-period EEG measurement during the thinking period (0-1.5 minutes) and resting period (1.5-3 minutes). The EEG frequency contour shows an obvious beta brain wave at 10-20 Hz only during the thinking period (FIG. 32). As a brain activity signature for alertness, concentration, and thinking, the beta wave in the EEG frequency contour collected by our electrodes differentiates thinking from resting. FIG. 33 shows the results of EEG brain wavelength contours and similar results are obtained on different shapes of earplugs and different individuals. To further improve spatial resolution and reduce the blurring effect due to volume conduction from multiple electrodes, tripolar EEG electrodes are designed and facilely fabricated by laser patterning to obtain the Laplacian signal (FIG. 34). As the second spatial derivative of the collected potentials, surface Laplacian EEG obtained from central and surrounding electrodes is reference-electrode-independent and reduces common noise, providing increased spatial selectivity and decreased mutual information in the measured EEG. Table 4 shows the performance comparison between the physiological sensor from this application and others in the literature. To conclude, our artifact-free, ultra-conformal, and multifunctional platform outperforms various other sensing materials.

TABLE 4
Performance comparison between the physiological sensor
from this application (bottom row) and others.

| Signals | Materials | Key Features | Limitations |
|---|---|---|---|
| ECG | Polymer, | Stretchable, | Requires tapes |
| | Ag NWs | stable | for fixing |
| ECG | Laser-induced | Fast fabrication | High signal to |
| | graphene | | noise ratio |
| ECG/EMG | Cu—PI—Au-PDMS | Low cost and | Weak |
| | | scalable | interfacial |
| | | | adhesion |
| ECG/EMG | PDMS, | Breathable, | High cost of |
| | PEDOT:PSS | long-term | materials |
| ECG/EMG/EEG | Graphene, | Conformal, | High cost of |
| | PEDOT:PSS | ultra-thin | materials |
| ECG/EMG | Ag, PEDOT:PSS | Conformal, | High cost of |
| | | drawn-on-skin | materials |
| ECG/EMG/EEG | Montmorillonite, | Artifact-free, | One-time use |
| | PVA, | conformal, | |
| | Ag/Cu@Cu NPs | multifunctional | |

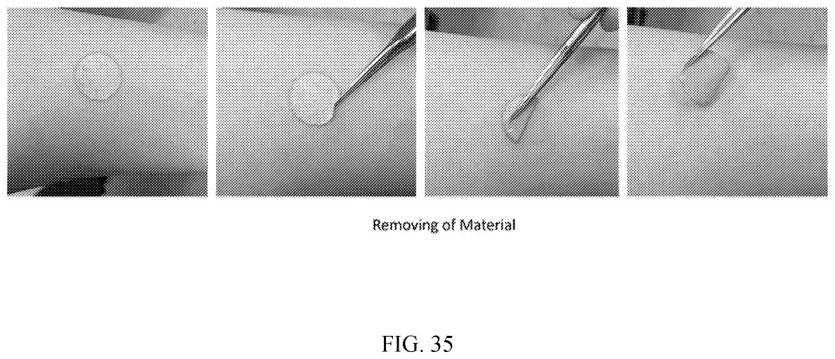

In summary, this example presents a class of conformal stretchable skin bioelectronics based on low-cost and facile laser patterning of a nanocomposite thin film for motion artifact-free sensing of electrophysiological and other biophysical signals. The nanocomposite thin film can be triggered by water molecules to result in partial dissolution and local deformation at the sensor/skin interface. Combined with the high conductive and stretchable properties, the resulting on-skin electrophysiology (EP) sensors exhibit reduced contact impedance and high signal quality even during mechanical deformations such as compression or stretching. The EP signals collected from this conformal device as a human-machine interface can be used for gesture recognition and game control. Used as stretchable and conductive interconnects, the patterned conformal thin film can facilely integrate the other commercial COTS chips for extended sensing and processing capabilities. The material can also be easily removed after use (FIG. 35). The concept is showcased in an integrated oximeter to measure pulse rate and blood oxygen saturation. The design concepts and application demonstrations of the multifunctional conformal device platform can also be adapted for other biophysical and biochemical sensors for motion artifact-free monitoring for the practical use of the next-generation wearable electronics. While the current sensor materials are disposable after use, it would be of high interest to exploit the possibility of potentially recycling and reusing these materials in future studies for sustainable applications.

It should be understood that modifications to the embodiments disclosed herein can be made to meet a particular set of design criteria. For instance, the number of or configuration of components or parameters may be used to meet a particular objective.

It will be apparent to those skilled in the art that numerous modifications and variations of the described examples and embodiments are possible in light of the above teachings of the disclosure. The disclosed examples and embodiments are presented for purposes of illustration only. Other alternative embodiments may include some or all of the features of the various embodiments disclosed herein. For instance, it is contemplated that a particular feature described, either individually or as part of an embodiment, can be combined with other individually described features, or parts of other embodiments. The elements and acts of the various embodiments described herein can therefore be combined to provide further embodiments.

It is the intent to cover all such modifications and alternative embodiments as may come within the true scope of this invention, which is to be given the full breadth thereof. Additionally, the disclosure of a range of values is a disclosure of every numerical value within that range, including the endpoints. Thus, while certain exemplary embodiments of the device and methods of making and using the same have been discussed and illustrated herein, it is to be distinctly understood that the invention is not limited thereto but may be otherwise variously embodied and practiced within the scope of the following claims.

What is claimed is:

1. A method of forming an electrode, the method comprising:
preparing a nanocomposite solution comprising nanoparticles dispersed within a polymeric solution;
depositing the nanocomposite solution onto a substrate to form a film;
patterning the film via laser scribing to form the electrode; and
optionally heating the electrode.

2. The method of claim 1, wherein the nanoparticles are core-shell nanoparticles comprising a core formed from a first metal and a shell formed from a second metal, and
wherein the first metal and the second metal are selected from the group consisting of silver, gold, platinum, palladium, titanium, copper, and zinc.

3. The method of claim 2, wherein the first metal is silver and the second metal is copper.

4. The method of claim 2, wherein the polymeric solution comprises one or more polymeric components selected from the group consisting of polyvinyl alcohol, ethylene vinyl alcohol, polyvinylpyrrolidone, polyethylene glycol, vinyl acetate ethylene, poly(acrylic acid), polyacrylic acid sodium salt, carboxymethyl cellulose, hydroxypropyl methylcellulose, and mixtures thereof.

5. The method of claim 4, wherein the polymeric solution comprises polyvinyl alcohol and polyethylene glycol.

6. The method of claim 4, wherein the polymeric solution further comprises one or more of the following:
one or more plasticizers,
one or more lubricants,
one or more humectants, and
one or more reinforcing fillers.

7. The method of claim 4, wherein the polymeric solution further comprises:
one or more components selected from the group consisting of glycerol, propylene glycol, ethylene glycol, sorbitol, polyethylene glycol, xylitol, and mannitol; and
one or more components selected from the group consisting of montmorillonite, kaolin, halloysite nanotubes, laponite, and silica nanoparticles.

8. The method of claim 7, wherein the polymeric solution further comprises glycerol and montmorillonite.

9. The method of claim 1, wherein the nanocomposite solution comprises 50-90 wt % of the nanoparticles, based on the total weight of the nanocomposite solution.

10. An electrode formed from the method of claim 1.

11. The electrode of claim 10, wherein one or more components of a polymeric matrix partially dissolves in the presence of water.

12. The electrode of claim 10, wherein the electrode has a sheet resistance at or below 1.2 Ω/sq upon stretching the electrode up to 30%.

13. A system comprising:
a sensing device comprising at least one electrode according to claim 6, wherein the at least one electrode is configured to collect electrophysiological data; and
an input/output device configured to receive the electrophysiological data from the sensing device.

14. The system of claim 13, further comprising:
a central computer device configured to receive the electrophysiological data from the sensing device and/or from the input/output device.

15. The system of claim 13, wherein the electrophysiological data comprises one or more selected from the group consisting of electrocardiograms (ECG) signals, electromyograms (EMG) signals, and electroencephalograms (EEG) signals.

16. A system comprising:
a sensing device comprising at least one electrode according to claim 6, wherein the at least one electrode is configured to collect data; and
an input/output device configured to receive the data from the sensing device,
wherein the data is selected from the group consisting of temperature data, humidity data, pulse rate, blood oxygen saturation, and combinations thereof.

17. The system of claim 16, further comprising:
a central computer device configured to receive the data from the sensing device and/or from the input/output device.
